(19) United States
(12) Patent Application Publication
(10) Pub. No.: US 2016/0136352 A1
(43) Pub. Date: May 19, 2016

(54) MEDICAL ORGANIZING EQUIPMENT

(71) Applicant: SAFE CARE TOOLS, LLC, Xenia, OH (US)

(72) Inventors: Revonna Smith, Xenia, OH (US); Peter Kosanovich, Algonquin, IL (US)

(21) Appl. No.: 14/823,055

(22) Filed: Aug. 11, 2015

Related U.S. Application Data
(60) Provisional application No. 62/080,465, filed on Nov. 17, 2014.

(51) Int. Cl. A61M 5/00; B65D 1/36

(52) U.S. Cl. CPC A61M 5/002; B65D 1/36

(57) ABSTRACT

An organizational system is disclosed, which comprises syringe compartments and vial compartments. Each of the vial compartments corresponds to one of the syringe compartments. The syringe compartments and vial compartments are designed to hold syringes and vials in an organized manner.

### CROSS REFERENCE TO RELATED APPLICATIONS

This application claims the benefit of U.S. provisional patent application Ser. No. 62/080,465, filed on 2014 Nov. 17, by Smith, and having the title “Organizing Equipment, Medication, and Other Items in Surgical Environments,” which is incorporated herein by reference in its entirety.

### BACKGROUND

1. Field of the Disclosure

The present disclosure relates generally to medical devices and, more particularly, to medical organizing equipment.

2. Description of Related Art

There are approximately 104 million inpatient and outpatient surgical procedures performed in the United States every year. Given the volume of surgical procedures, there are ongoing efforts to reduce errors that occur in operating rooms, emergency rooms, intensive care units, military environments, and other environments where medications are prepared or administered.

### SUMMARY

The present disclosure provides organizing systems for medical environments.

Some embodiments, among others, comprise syringe compartments and vial compartments. Each of the vial compartments corresponds to one of the syringe compartments. The syringe compartments and vial compartments are designed to hold syringes and vials in an organized manner.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A is a diagram showing one embodiment of a medical organizing tray.

FIG. 1B is a diagram showing a top view of the tray of FIG. 1A.

FIG. 1C is a diagram showing a front view of the tray of FIG. 1A.

FIG. 1D is a diagram showing a side view of the tray of FIG. 1A.

FIG. 2 is a diagram showing another embodiment of an organizing tray.

FIG. 3 is a diagram showing one embodiment of a tray with syringes.

FIG. 4 is a diagram showing one embodiment of a tray with syringes and vials.

FIG. 5 is a diagram showing one embodiment of a tray and a platform for the tray.

FIG. 6 is a diagram showing yet another embodiment of an organizing tray.

FIG. 7 is a diagram showing yet another embodiment of an organizing tray.

### DETAILED DESCRIPTION OF THE EMBODIMENTS

Statistics relating to operating room errors are staggering. There are approximately 104 million inpatient and outpatient surgical procedures performed in the United States every year. One medication error occurs in every 133 anesthetics administered, with higher error rates occurring in intensive care units, operating rooms, and emergency departments where pressure is significantly higher than on a regular floor. Of those errors, one out of every 250 is fatal. A Canadian Closed Claim Study showed that the most common cause (52%) of all litigation claims was due to medication errors. A survey by the Canadian Anesthesiologist Society revealed that 85% of respondents have experienced at least one drug error or near miss. Moreover, the inventor has recognized that misidentification of a syringe is a common cause of error. Recent estimates reveal that 81% of all hospital medication errors occur in the operating room and post-anesthesia care units. These errors can lead to significant patient complications and even death. Some of these errors can be attributed to cluttered arrangement of medications used by anesthesiologists or other anesthesia providers. Requests by prominent anesthesiology societies for improvements in medication delivery with regards to patient safety in the operating room have gone unanswered, thereby leaving a long-felt need as appreciated by those having ordinary skill in the art.

The disclosed embodiments seek to meet the long-felt need by providing organizing equipment for use in surgical environments or other environments where medications are prescribed, prepared, administered, and transported. Generally, the organizing equipment comprises a syringe tray in combination with a medication tray. The syringe tray holds multiple syringes and the medication tray holds different medications that correspond to the syringes in the syringe tray. Providing an organizing tray for the medications and syringes results in reduction (if not elimination) of clutter, which in turn results in fewer errors and greater efficiency in these environment.

Having provided an overview of the inventive concept, reference is now made in detail to the description of the embodiments as illustrated in the drawings. While several embodiments are described in connection with these drawings, there is no intent to limit the disclosure to the embodiment or embodiments disclosed herein. On the contrary, the intent is to cover all alternatives, modifications, and equivalents.

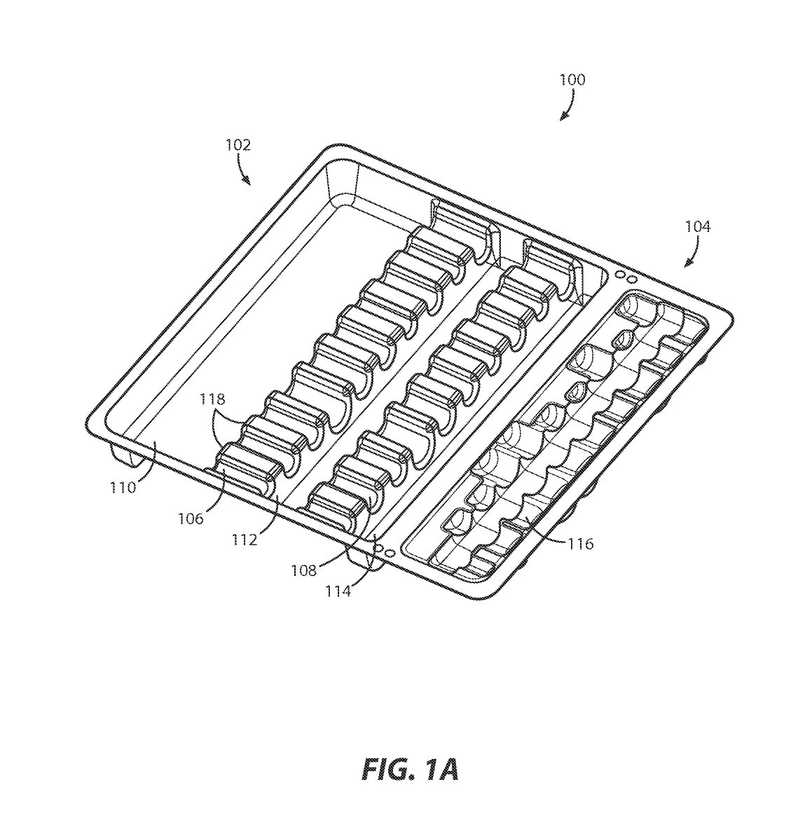

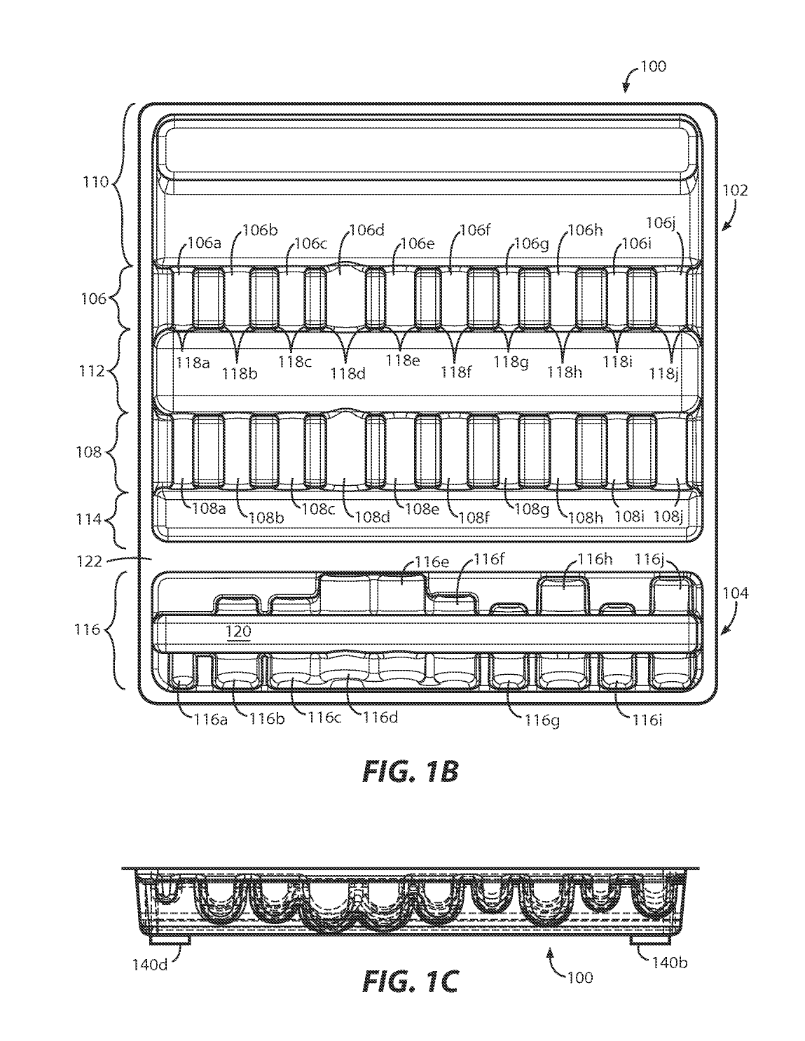

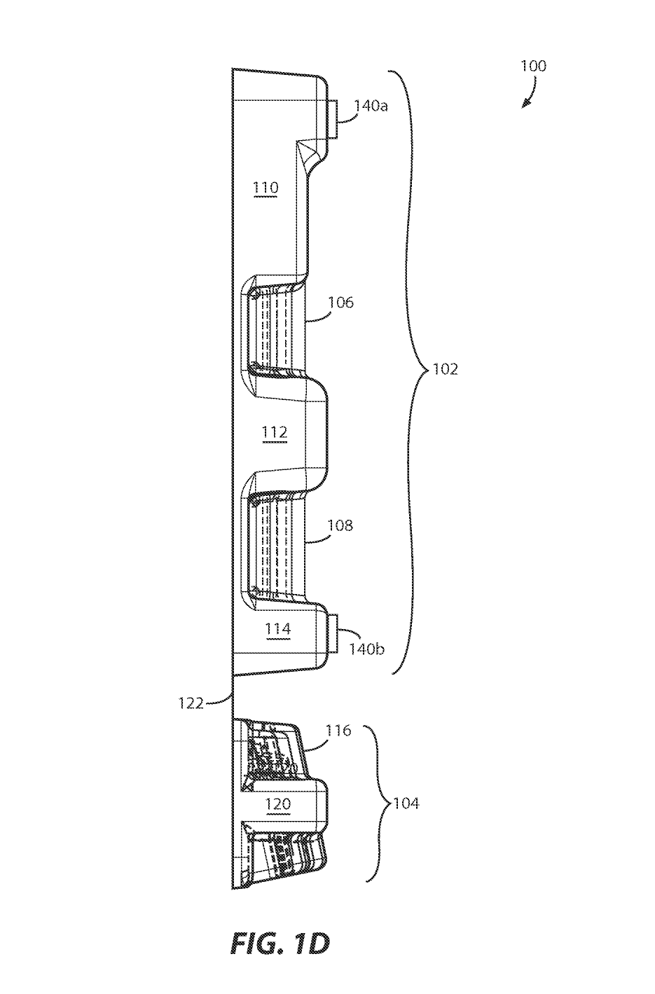

FIGS. 1A through 1D (collectively, FIG. 1) show various viewpoints for one embodiment of a medical organizing tray 100. As shown in FIG. 1, the tray 100 comprises two general portions, namely, a syringe tray 102 and a vial tray 104. The syringe tray 102 comprises syringe compartments that have recesses 106a-106j (collectively, 106) with protrusions 118a-118j (collectively, 118) on each side of each recess 106. The recesses 106 have dimensions that correspond to a barrel of a syringe, such as, for example, a three milliliter (3 ml) syringe, a five (5) ml syringe, a ten (10) ml syringe, or a twenty (20) ml syringe, which are standard syringe sizes used in operating rooms or other similar medical environments. The protrusions 118 (one on the left side of the recess and one on the right side of the recess) work cooperatively to releasably secure the barrel of the syringe within the recess 106. In other words, the protrusions 118 are dimensioned so as to secure syringes within the recess while also permitting relatively easy removal of the syringe by a medical professional. Optionally, the syringe tray 102 may also include an additional set of recesses 108a-108j (collectively 108) having protrusions in an analogous manner to that set out above with reference to recesses 106. To this end, the syringe tray 102 also comprises spaces 110, 112, 114 that allow room for fingers to grasp any syringes that are held within the recesses 106 (and optionally recesses 108).

The vial tray 104 comprises vial compartments 116a-116j (collectively, 116). Each of the vial compartments 116 corresponds to one of the syringe compartments 106. Furthermore, each vial compartment 116 has recesses that correspond to dimensions of a medication vial, thereby permitting the vial tray 104 to releasably secure medication vials, similar to how the syringe tray 102 releasably secures syringes. As described in greater detail below, the vial tray 104 is designed to hold various medications that are used during surgery, such as, for example, sedatives, pain relievers, general and local anesthetics, muscle relaxants, antiemetic agents, and various other drugs based on a patient's needs during surgery or during other environments that require organization and stabilization of medications. The vial tray 104 also includes a space 120 that allows room for fingers to individually grasp any vials that are held within the vial compartments 116. In the embodiment of FIG. 1, the syringe tray 102 is separated from the vial tray 104 by a connector 122.

In one preferred embodiment, the syringe compartments 106 are oriented substantially parallel to each other. The parallel orientation provides better visual appeal as well as better organization of both the syringes and the vials. For some embodiments, the syringe tray 102 comprises magnets 140a-140d (collectively, 140) located on each corner at the bottom of the syringe tray 102. As described in greater detail below, the magnets allow for securing of the tray 100 to a metallic platform. However, it should be appreciated that the magnets 140 can be placed in other locations to improve functionality or provide better securing of the tray 100 to the platform.

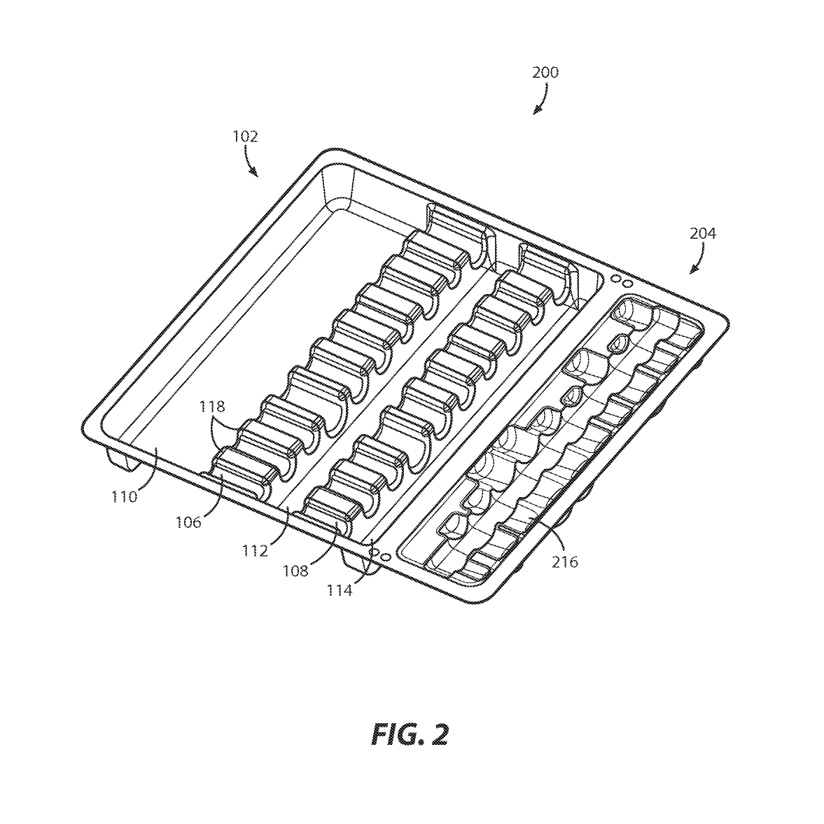

FIG. 2 is a diagram showing another organizing tray 200. Similar to the tray 100 of FIG. 1, this embodiment of the tray 200 comprises a syringe tray 102 and a vial tray 204. Unlike the embodiment of FIG. 1, the vial tray 204 comprises angled vial compartments 216 to hold medication vials. By holding medication vials at an angle, the angled vial compartments 216 permit easier reading of the medication vial labels for the medical professional. For some embodiments, the vial compartments 216 are dimensioned to friction fit medication vials. For other embodiments, the vial compartments 216 may include protrusions similar to those found on the syringe tray 102.

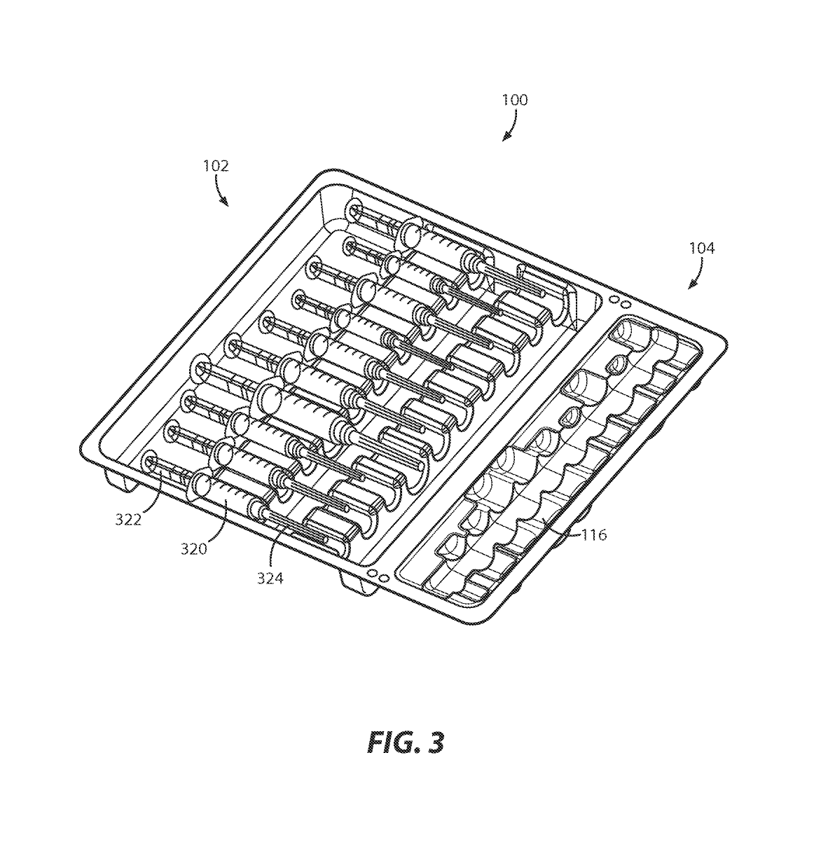

FIG. 3 is a diagram showing the tray 100 of FIG. 1 in which the tray 100 holds various syringes. As is known by those having ordinary skill in the art, syringes have three (3) main components, namely, a barrel 320, a plunger 322, and a needle or a cap 324. As described with reference to FIG. 1, the barrels 320 of the syringes are held securely within the recesses 106 by the protrusions 118 on either side of the recesses 106. It should be appreciated that, even though a set of two (2) protrusions are shown in the drawings, some embodiments function with a single protrusion on one side of the recess (as opposed to two protrusions on either side of the recess). In the particular embodiment of FIG. 3, each of the syringes corresponds to a particular medication that is used during surgery. In one preferred embodiment, the syringe compartments hold the following syringes: (a) 3 ml syringe for midazolam; (b) 5 ml syringe for fentanyl; (c) 3 ml syringe for lidocaine; (d) 20 ml syringe for propofol; (e) 10 ml syringe for succinylcholine; (f) 5 ml syringe for rocuronium or cisatracrium (both non-depolarizing muscle relaxants); (g) 3 ml syringe for glycopyrrolate; (h) 5 ml syringe for neostigmine; (i) 3 ml syringe for ondansetron (or Zofran®); and (j) an empty syringe (which can be used at the provider's discretion, for example, to be used with decadron, ephedrine, neosynephrine, etc.).

In one preferred embodiment, the syringes (meaning the barrels, plungers, needle caps, or any combinations thereof) are also color-coded can be colored. The color coding reduces the possibility of the medical professional selecting the wrong medication. For example, the midazolam syringe can be orange; the fentanyl (or other narcotic) syringe, blue; the lidocaine syringe, gray; the propofol syringe, yellow; the succinylcholine syringe, a combination of red and black striped; the glycopyrrolate syringe, green; the neostigmine syringe, red and white striped; and the ondansetron syringe (which currently has no designated color label). As an alternative, each of the recesses 106 in the syringe tray 102 can be color-coded.

Those having skill in the art will appreciate that different combinations of syringes and medications can be used, depending on a patient's specific needs or based on a particular surgical procedure or situational need or environment. Thus, while particular medications are recited with reference to FIG. 3, it should be appreciated that different sedatives, pain relievers, anesthetics, muscle relaxants, etc., can be used in place of the specifically-recited medications, above. Furthermore, it should be appreciated that, for some embodiments, it is not necessary to fill all ten (10) syringe compartments 106. For example, for some procedures it may be necessary to use only two (2) or three (3) different medications. Consequently, it should be appreciated that the syringe tray 102 can be configured for anywhere between two (2) to ten (10) or more syringe compartments 106. Also, while specific color-coding is disclosed for clarity based in industry convention, it should be noted that the color coding can be varied to use different color combinations. At bottom, the syringe compartments 106 provide an organized manner in which different syringes for different medications are made readily accessible to the medical professional in an uncluttered arrangement.

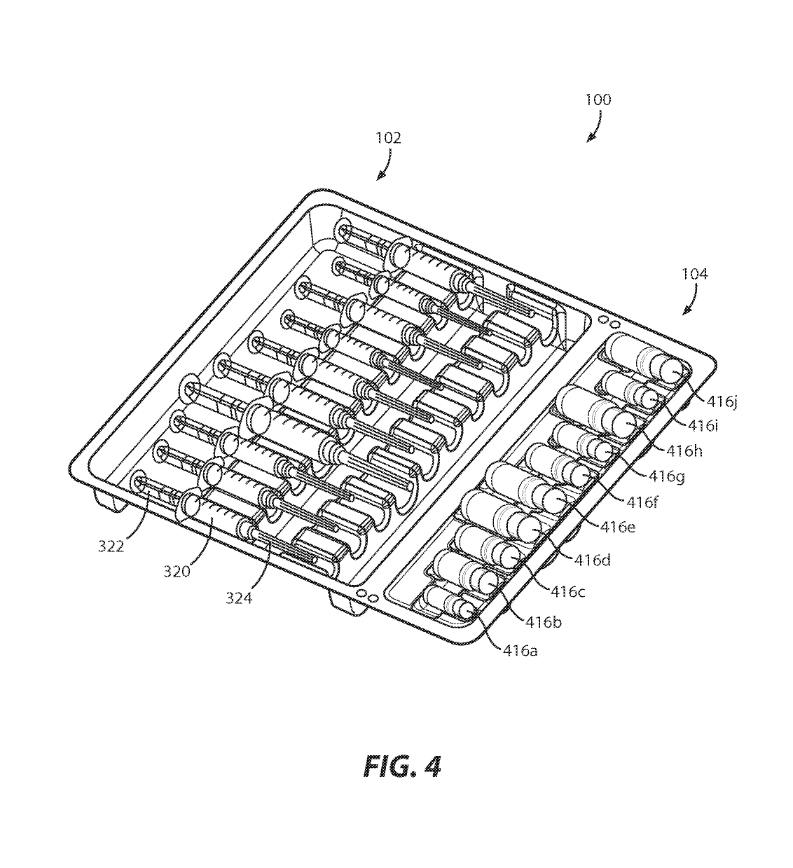

FIG. 4 is a diagram showing the tray 100 of FIG. 1 with both syringes and various medication vials 416a-416j (collectively, 416). Continuing from the specific embodiment described with reference to FIG. 3, the vials 416 corresponds to the syringes, for example: (a) the first vial compartment holds a vial of midazolam 416a, which corresponds to the first syringe compartment 106a that holds the syringe for midazolam; (b) the second vial compartment holds a vial of fentanyl 416b, which corresponds to the second syringe compartment 106b that holds the syringe for fentanyl; and so on for the remaining medication vials (e.g., lidocaine 416c, propofol 416d, succinylcholine 416e, rocuronium (or cisatracurium) 416f, glycopyrrolate 416g, neostigmine 416h, ondansetron (or Zofran®) 416i), etc.). By arranging each vial 416 to correspond to its syringe, the tray 100 reduces clutter, thereby reducing the possibility of error in the operating room or other similar environment. Additionally, by using such an organizational tray as part of a holistic system for securing, preparing, and administering medications, the possibility of error can be further reduced.

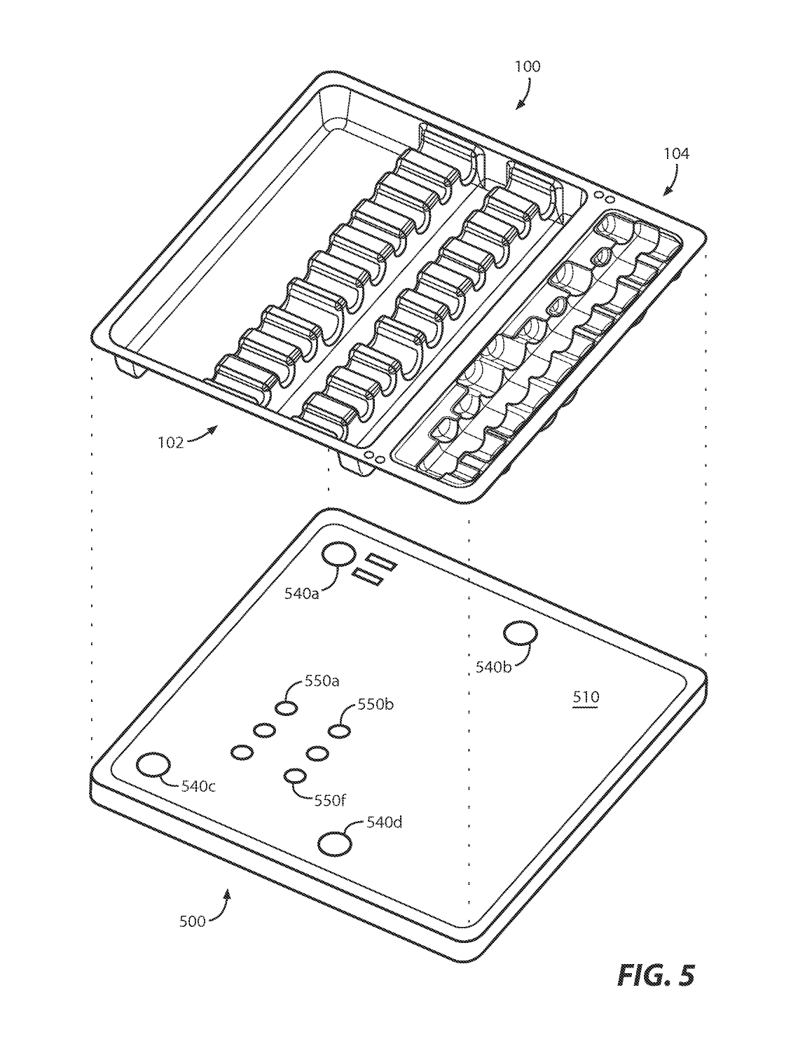

FIG. 5 is a diagram showing the tray 100 of FIG. 1 and a platform 500 for mounting the tray 100. Although it is possible for the tray 100 to sit on any flat surface in the operating room, it is preferable to provide a dedicated surface for the tray 100. This is because, without a dedicated surface, it is possible to bump the tray 100 and drop the tray 100 to an unsterile floor. To avoid this, the embodiment of FIG. 5 shows a platform 500 on which to mount or secure the tray 100. As shown in FIG. 5, the platform 500 comprises magnets 540a-540d (collectively, 540), each of which are located on the platform 500 where they correspond to the magnets 140 on the tray 100. Functionally, the platform magnets 540 will mate to the tray magnets 540, thereby preventing the tray 100 from being jostled around or falling to the floor. It should be appreciated that only one set of magnets 140 (on the tray 100) is needed if the platform 500 comprises a ferromagnetic metallic surface 510. Alternatively, the magnets 540 can be mounted to the platform, while ferromagnetic metal studs are mounted on the tray, which may reduce the cost of manufacturing the tray 100.

For some embodiments, the tray 100 is semi-transparent and the platform 500 comprises lights 550a-550f (collectively, 550) on the surface 510. The lights (preferably light emitting diodes (LED)) provide back-lighting to the tray 100 thereby allowing the medical professional to more-easily distinguish between the different syringes and medication vials. Also, if the lights 550 are positioned to correspond with the recesses 106 on the syringe tray 102, then different colored LEDs can be used to distinguish between the different medications. In other words, rather than providing different syringe colors or different colors on the tray, which may be more expensive, using different colored LEDs 550 on the platform 500 can provide the same color-coding effect without added manufacturing costs.

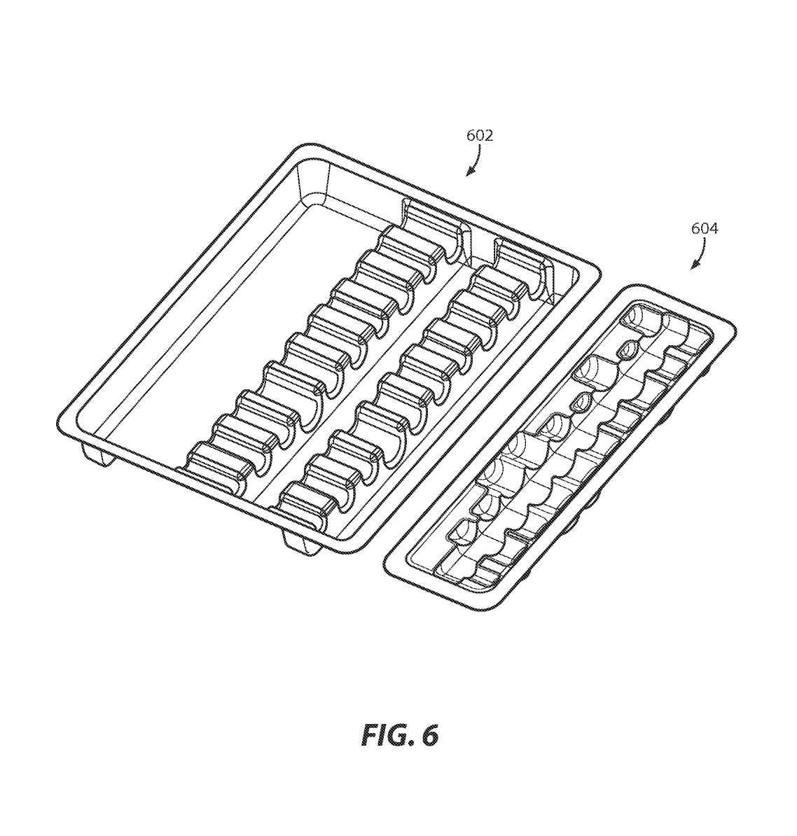

FIG. 6 is a diagram showing yet another embodiment of an organizing tray. Unlike FIGS. 1 and 2, which showed a single-unit tray having both a syringe tray 102 and an vial tray 104, 204, the embodiment of FIG. 6 shows the syringe tray 602 being separate from the vial tray 604. Although separating the vial tray 604 from the syringe tray 602 provides added flexibility, the preferred approach is to have a single tray 100, 200 that holds both the syringes and the vials. This is because there is no doubt that the medications and the syringes are aligned in the single tray 100, 200, while it is possible for the vials and the syringes to become offset in the two-tray embodiment of FIG. 6.

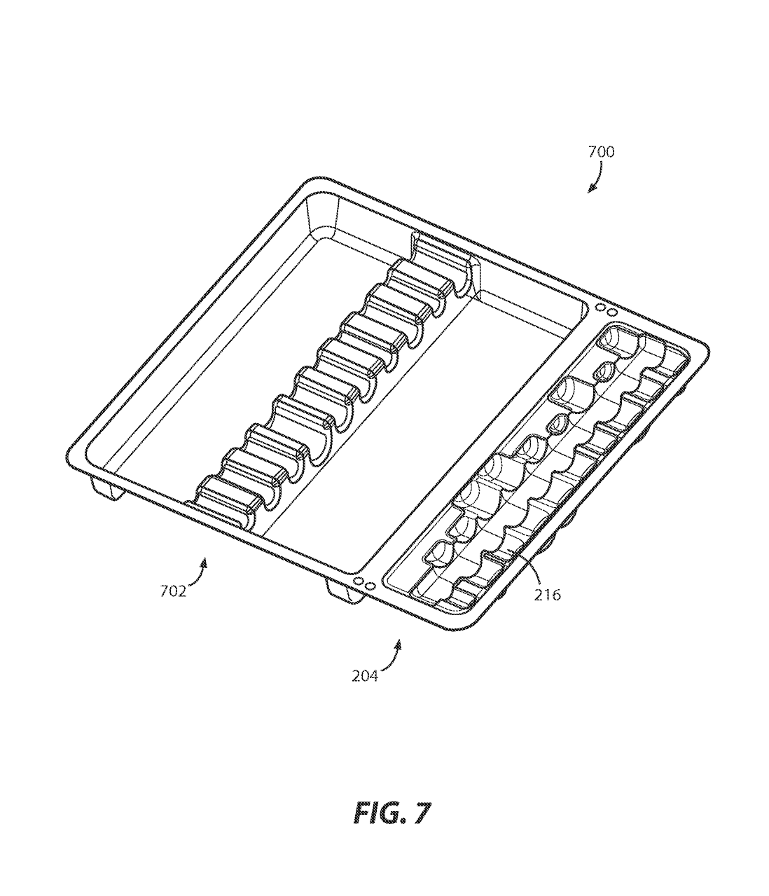

FIG. 7 is a diagram showing yet another embodiment of an organizing tray 700. Unlike the embodiments of FIGS. 1 and 2, which show two (2) sets of recesses 106, 108 to hold syringes, the embodiment of FIG. 7 shows a single set of recesses that are located approximately in the middle of the syringe tray 702. The centralized location of the recesses in FIG. 7 allows the syringe tray 702 to hold the syringes with the needles facing toward the vial tray 204 or, in the alternative, to hold the syringes with the needles facing away from the vial tray 204.

Those having skill in the art will appreciate that the tray 100, 200, 602, 604, 700 can be manufactured using known injection-molding processes, plastic welding processes, or other known plastic manufacturing processes. Also, in order to better enable those having skill in the art to manufacture the tray 700, the dimensions of the tray 700 are, preferably, approximately 13 inches (approximately 330 millimeters (mm)) wide (with the spaces between each of the syringe compartments 106 being approximately equal), approximately 13.5 inches (approximately 343 mm) in length for the combination of the syringe tray 702 and the vial tray 204 (with the syringe tray 702 being approximately 10.25 inches (approximately 260 mm) and the vial tray 204 being approximately 3.25 inches (approximately 83 mm)). The preferable depth of the tray, at its deepest point, is approximately 1.56 inches (approximately 40 mm). For embodiments in which the vial tray 204 is angled, the preferred angle is anywhere between approximately 15 degrees and approximately 45 degrees. Even though these specific dimensions are provided for clarity, it should be appreciated that different dimensions are contemplated, depending on the number of syringe compartments and the sizes of the syringes, the number of vial compartments and vials, or any combinations of vials and syringes.

As shown in the embodiments of FIGS. 1A through 7, providing equipment that reduces clutter in the operating room provides a corresponding improvement by reducing operating room errors that are attributable to anesthesiologists, anesthesia providers, or other medical professionals. Additionally, to the extent that the trays 100, 200, 700 can be mass produced, preferred embodiments of the trays 100, 200, 700 are manufactured to be disposable or recyclable.

Although exemplary embodiments have been shown and described, it will be clear to those of ordinary skill in the art that a number of changes, modifications, or alterations to the disclosure as described may be made, and additional advantages are realizable from the disclosed embodiments. For example, keeping the tray as a single unit (rather than one separate unit for syringes and another separate unit for vials) simplifies workflow. Furthermore, one will appreciate that an additional visible indentation, protrusion, or other type of separator can be included between the various compartments to visually separate the different syringes and vials from each other. Also, the name (or other designation) of any specific medication is embeddable into the tray itself so that individuals loading the trays have a point of reference for where each medication belongs (e.g., “M” for midazolam, along with 3 cc for syringe size; “F” with “5 cc” for fentanyl with 5 cc; “L” with “3 cc” for lidocaine; “P” with 20 cc for propofol; “G” with 3 cc for glycopyrolate; etc.). For other embodiments, the tray can be pre-loaded with various pre-labeled syringes and needles and, also, pre-labeled with its corresponding medication vial. Also, the syringe tray can be sterilized and sealed so that the medication vials can be selectively loaded onto the tray without any risk of contamination to the syringes. For yet other embodiments, medication vials that are selectively loaded onto the tray can be secured into the tray, for example, by using zip ties or other securing mechanisms. In other embodiments, the size of the syringe trays may be uniformly manufactured and different size syringes can be accommodated by using compressible materials that can be modularly added to the trays to account for the different sizes. It should also be appreciated that the tray can be manufactured from recyclable or compostable materials, thereby making the trays environmentally friendly. All such changes, modifications, and alterations should therefore be seen as within the scope of the disclosure.

What is claimed is:

1. An organizing tray for surgical environments, the tray comprising:
(a) syringe compartments, each syringe compartment comprising;
(a1) a recess having dimensions that correspond to a barrel of a syringe, the syringe being one selected from the group consisting of:
(a1A) a three milliliter (3 ml) syringe;
(a1B) a five (5) ml syringe;
(a1C) a ten (10) ml syringe; and
(a1D) a twenty (20) ml syringe;
(a2) a left protrusion located on a left side of the recess; and
(a3) a right protrusion located on a right side of the recess, the right protrusion and the left protrusion for cooperatively securing the barrel of the syringe, the right protrusion and the left protrusion further for releasably securing the barrel of the syringe within the recess; and
(b) vial compartments, each vial compartment corresponding to a syringe compartment, each vial compartment having dimensions that correspond to dimensions of a medication vial, each vial compartment for releasably securing the medication vial, the medication vial being one selected from the group consisting of:
(b1) a propofol vial;
(b2) a lidocaine vial;
(b3) a fentanyl vial;
(b4) a midazolam vial;
(b5) a succinylcholine vial;
(b6) a rocuronium vial;
(b7) a glycopyrrolate vial;
(b8) a neostigmine vial; and
(b9) an ondansetron vial.

2. The tray of claim 1, further comprising:
(c) a connector located between the syringe compartments and the vial compartments.

3. The tray of claim 1 being semi-transparent.

4. The tray of claim 1, the syringe compartments being substantially parallel to each other, the syringe compartments comprising:
a first syringe compartment for a 3 ml syringe for midazolam;
a second syringe compartment for a 5 ml syringe for fentanyl;
a third syringe compartment for a 3 ml syringe for lidocaine;
a fourth syringe compartment for a 20 ml syringe for propofol;
a fifth syringe compartment for a 10 ml syringe for succinylcholine;
a sixth syringe compartment for a 5 ml syringe for rocuronium;
a seventh syringe compartment for a 3 ml syringe for glocopyrolate;
an eighth syringe compartment for a 5 ml syringe for neostigmine; and
a ninth syringe compartment for a 3 ml syringe for ondansetron.

5. The tray of claim 4, the syringe compartments further comprising:
a tenth syringe compartment for an empty syringe.

6. The tray of claim 1, the vial compartments being substantially parallel to each other, the vial compartments comprising:
a first vial compartment for holding a vial of midazolam;
a second vial compartment for holding a vial of fentanyl;
a third vial compartment for holding a vial of lidocaine;
a fourth vial compartment for holding a vial of propofol;
a fifth vial compartment for holding a vial of succinylcholine;
a sixth vial compartment for holding a vial of rocuronium;
a seventh vial compartment for holding a vial of glocopyrolate;
an eighth vial compartment for holding a vial of neostigmine; and
a ninth vial compartment for holding a vial of ondansetron.

7. A system comprising:
a first syringe compartment, comprising:
a first recess; and
a first protrusion located on one side of the first recess, the first protrusion for releasably securing within the first recess a first syringe for propofol; and
a second syringe compartment oriented substantially parallel to the first syringe compartment, the second syringe compartment comprising:
a second recess; and
a second protrusion located on one side of the second recess, the second protrusion for releasably securing within the second recess a second syringe, the second syringe being one selected from the group consisting of:
a syringe for lidocaine;
a syringe for fentanyl;
a syringe for midazolam;
a syringe for succinylcholine;
a syringe for rocuronium;
a syringe for glycopyrrolate;
a syringe for neostigmine; and
a syringe for ondansetron.

8. The system of claim 7, the second protrusion for releasably securing within the second recess the syringe for lidocaine, the system further comprising:
a third syringe compartment oriented substantially parallel to the first syringe compartment, the third syringe compartment further being oriented substantially parallel to the second syringe compartment, the third syringe compartment comprising:
a third recess; and
a third protrusion located on one side of the third recess, the third protrusion for releasably securing within the third recess a third syringe, the third syringe being one selected from the group consisting of:
a syringe for fentanyl;
a syringe for midazolam;
a syringe for succinylcholine;
a syringe for rocuronium;
a syringe for glycopyrrolate;
a syringe for neostigmine; and
a syringe for ondansetron.

9. The system of claim 7, further comprising:
a first vial compartment corresponding to the first syringe compartment, the first vial compartment for releasably securing a vial of propofol; and
a second vial compartment corresponding to the second syringe compartment, the second vial compartment for releasably securing a second vial, the second vial being one selected from the group consisting of:
a vial of lidocaine;
a vial of fentanyl;
a vial of midazolam;
a vial of succinylcholine;
a vial of rocuronium;
a vial of glycopyrrolate;
a vial of neostigmine; and
a vial of ondansetron.

10. The system of claim 9, the second protrusion for releasably securing within the second recess the syringe for lidocaine, the second vial compartment for releasably securing the vial of lidocaine, the system further comprising:
a third syringe compartment oriented substantially parallel to the first syringe compartment, the third syringe compartment further being oriented substantially parallel to the second syringe compartment, the third syringe compartment comprising:
a third recess; and
a third protrusion located on one side of the third recess, the third protrusion for releasably securing within the third recess a third syringe, the third syringe being one selected from the group consisting of:
a syringe for fentanyl;
a syringe for midazolam;
a syringe for succinylcholine;
a syringe for rocuronium;
a syringe for glycopyrrolate;
a syringe for neostigmine; and
a syringe for ondansetron; and
a third vial compartment for releasably securing a third vial, the third vial being one selected from the group consisting of:
a vial of fentanyl;
a vial of midazolam;
a vial of succinylcholine;
a vial of rocuronium;
a vial of glycopyrrolate;
a vial of neostigmine; and
a vial of ondansetron.

11. An organizing system, comprising:
a first syringe compartment, comprising:
a first recess; and
a first protrusion located on one side of the first recess, the first protrusion for releasably securing within the first recess a first syringe for a first medication;
a second syringe compartment oriented substantially parallel to the first syringe compartment, the second syringe compartment comprising:
a second recess; and
a second protrusion located on one side of the second recess, the second protrusion for releasably securing within the second recess a second syringe for a second medication, the second medication being different from the first medication;
a third syringe compartment oriented substantially parallel to the first syringe compartment, the third syringe compartment further being oriented substantially parallel to the second syringe compartment, the third syringe compartment comprising:
a third recess; and
a third protrusion located on one side of the third recess, the third protrusion for releasably securing within the third recess a third syringe for a third medication, the third medication being different from the first medication, the third medication further being different from the second medication.

12. The system of claim 11, further comprising:
a first vial compartment corresponding to the first syringe compartment, the first vial compartment for releasably securing a vial of the first medication;
a second vial compartment corresponding to the second syringe compartment, the second vial compartment for releasably securing a vial of the second medication;
a third vial compartment corresponding to the third syringe compartment, the third vial compartment for releasably securing a vial of the third medication.

13. The system of claim 12:
the first vial compartment being angled;
the second vial compartment being angled; and
the third vial compartment being angled.

14. The system of claim 12:
the vial of the first medication being a vial of propofol;
the vial of the second medication being a vial of lidocaine; and
the vial of the third medication being a vial of fentanyl.

15. The system of claim 11:
the first syringe being a 20 ml syringe;
the second syringe being a 5 ml syringe; and
the third syringe being a 5 ml syringe.

16. The system of claim 11, further comprising:
a fourth syringe compartment oriented substantially parallel to the first syringe compartment, the fourth syringe compartment further being oriented substantially parallel to the second syringe compartment, the fourth syringe compartment further being oriented substantially parallel to the third syringe compartment, the fourth syringe compartment comprising:
a fourth recess; and
a fourth protrusion located on one side of the fourth recess, the fourth protrusion for releasably securing within the fourth recess a fourth syringe for a fourth medication, the fourth medication being different from the first medication, the fourth medication further being different from the second medication, the fourth medication further being different from the third medication.

17. The system of claim 16, the fourth syringe being one selected from the group consisting of:
a 3 ml syringe;
a 5 ml syringe;
a 10 ml syringe; and
a 20 ml syringe.

18. The system of claim 12, further comprising:
a fourth vial compartment for releasably securing a vial of the fourth medication, the fourth medication being one selected from the group consisting of:
propofol;
lidocaine;
fentanyl;
midazolam;
succinylcholine;
rocuronium;
glycopyrrolate;
neostigmine; and
ondansetron.

19. The system of claim 11:
the first syringe compartment corresponding to a first color;
the second syringe compartment corresponding to a second color, the second color being different from the first color; and
the third syringe compartment corresponding to a third color, the third color being different from the first color, the third color further being different from the second color.

20. The system of claim 11, further comprising:
means for magnetically coupling the first syringe compartment to a platform.
